(19) United States
(12) Patent Application Publication
(10) Pub. No.: US 2009/0163515 A1
(43) Pub. Date: Jun. 25, 2009

(54) Compounds Which Bind to the Active Site of Protein Kinase Enzymes

(75) Inventors: Veronique Birault, Essex (GB); C. John Harris, Kent (GB)

Correspondence Address: MARTIN SAVITZKY;Law Offices of Martin Savitzky Esq., PO BOX 1027, FORT WASHINGTON, PA, 19034-1027, US

(73) Assignee: Biofocus Discovery Ltd.

(21) Appl. No.: 10/561,914

(22) PCT Filed: Jul. 1, 2004
(86) PCT No.: PCT/GB2004/002849

§ 371 Date: Feb. 29, 2008

(30) Foreign Application Priority Data
Jul. 2, 2003 (GB) 0315494.5
Dec. 9, 2003 (GB) 0328497.3

(51) Int. Cl. A61K 31/4965; C07D 241/10; C07D 213/02; A61K 31/44; C07D 215/00; A61K 31/47; A61K 31/444; C07D 239/24; A61K 31/505; A61P 9/10; A61P 9/12

(52) U.S. Cl. 514/255.05; 544/405; 546/304; 514/352; 546/167; 514/314; 546/257; 514/334; 544/333; 514/256

(57) ABSTRACT

The present invention relates to a compound and a group of compounds which are inhibitors of Rho kinase (ROK, ROCK). In addition, the invention relates to methods of treatment and use of the compounds in the manufacture of a medicament for application to a number of therapeutic indications including cardiovascular disease (coronary vasospasm, hypertensive disease, arteriosclerosis), stroke, cancer, erectile dysfunction, asthma, osteoporosis, glaucoma and AIDS. The compounds can be used in screening programmes against protein kinases. The invention also provides methods for making compounds and libraries that include these compounds.

The present invention relates to a compound and a group of compounds capable of binding to the active site of protein kinase enzymes. In particular, the invention relates to a compound and a group of compounds which are inhibitors of a serine/threonine kinase more particularly Rho kinase (ROK, ROCK). In addition, the invention relates to methods of treatment and use of the compounds in the manufacture of a medicament for application to a number of therapeutic indications including cardiovascular disease (coronary vasospasm, hypertensive disease, arteriosclerosis), stroke, cancer, erectile dysfunction, asthma, osteoporosis, glaucoma and AIDS. The compounds can be used in screening programmes against protein kinases. The invention also provides methods for making compounds and libraries that include these compounds.

The Kinase Gene Family

Protein kinases are a family of enzymes that catalyse the phosphorylation of hydroxyl groups in proteins. Approximately 2% of the genes encoded by the human genome are predicted to encode protein kinases. The reversible phosphorylation of specific tyrosine, serine, or threonine residues on a target protein can dramatically alter its function in several ways including activating or inhibiting enzymatic activity; creating or blocking binding sites for other proteins; altering subcellular localisation or controlling protein stability. Consequently protein kinases are pivotal in the regulation of a wide variety of cellular processes, including metabolism, cell proliferation, differentiation and survival. Of the many different cellular functions know to require the actions of protein kinases, some represent targets for therapeutic intervention for certain disease states.

One of the principal mechanisms by which cellular regulation is effected is through the transduction of extracellular signals across the membrane that, in turn, modulate biochemical pathways within the cell. Protein phosphorylation represents one course by which intracellular signals are propagated from molecule to molecule resulting finally in a cellular response. These signal transduction cascades are highly regulated and often overlapping as evidenced by the existence of many protein kinases as well as phosphatases. It is currently believed that a number of disease states and/or disorders are a result of either aberrant activation or functional mutations in the molecular components of kinase cascades. In humans, protein tyrosine kinases are known to have a significant role in the development of many disease states including diabetes, cancer and have also been linked to a wide variety of congenital syndromes. Serine threonine kinases also represent a class of enzymes, inhibitors of which are likely to have relevance to the treatment of cancer, diabetes and a variety of inflammatory cardiovascular disorders and AIDS.

Three potential mechanisms for inhibition of protein kinases have been identified thus far. These include a pseudo-substrate mechanism, an adenine mimetic mechanism and the locking of the enzyme into an inactive conformation by using surfaces other than the active site. The majority of inhibitors identified/designed to date act at the ATP-binding site. Such ATP-competitive inhibitors have demonstrated selectivity by virtue of their ability to target the more poorly conserved areas of the ATP-binding site.

Modulation of protein kinase activity therefore represents an attractive area for the design of new therapeutic agents. Protein kinases therefore represent a targeted intervention point in the treatment of a wide range of diseases.

Rho Kinases (ROK)

The Rho family of small GTP binding proteins contains at least 10 members including Rho A-E and G, Rac 1 and 2, Cdc42, and TC10. The effector domains of RhoA, RhoB, and RhoC have the same amino acid sequence appear to have similar intracellular targets. Rho kinase operates as a primary downstream mediator of Rho and exists as two isoforms α (ROCK2) and β (ROCK1).

ROK has a catalytic (kinase) domain in its N-terminal domain, a coiled-coil domain in its middle portion, and a putative pleckstrin-homology (PH) domain in its C-terminal domain. The Rho-binding domain of ROK is localized in the C-terminal portion of the coiled-coil domain and the binding the GTP-bound form of Rho results in enhancement of kinase activity. Numerous substrates of this kinase have been identified: myosin-binding subunit of myosin light-chain phosphatase; ERM (ezrin, radixin, moesin); adducin; intermediate filament (vimentin); the Na⁺-H⁺-exchanger, and LIM-kinase.

The Rho/Rho-kinase-mediated pathway plays an important role in the signal transduction initiated by many agonists, including angiotensin II, serotonin, thrombin, endothelin-1, norepinephrine, platelet-derived growth factor, ATP/ADP and extracellular nucleotides, and urotensin II. Through the modulation of its target effectors/substrates ROK plays an important role in various cellular functions including smooth muscle contraction, actin cytoskeleton organization, cell adhesion and motility and gene expression.

Therapeutic Potential of ROK Inhibitors

The apparent contribution of ROK to the pathogenesis of certain disorders has highlighted this kinase as a target for therapeutic intervention in a number of disease areas. The first generation ROK inhibitor, fasudil and the more recent Y-27632 compound has provided proof of concept in a variety of model systems.

Rho-kinase inhibitors have potential utility for the treatment of disorders caused by vascular smooth muscle hyper-constriction, including cerebral vasospasm, coronary vasospasm and hypertension. The beneficial effects of fasudil in the inhibition of cerebral and coronary vasospasm have been documented and there is accumulating evidence that ROK is involved in the pathogenesis of such events. ROK levels of expression and activity are significantly enhanced prior to development of symptoms in spontaneously hypertensive rats suggesting that this kinase is also involved in the pathogenesis of hypertension. Furthermore, short-term administration of Y-27632 preferentially reduces systemic blood pressure in various models of systemic hypertension.

By virtue of ROK's role in mediating a number of cellular functions perceived to be associated with the pathogenesis of arteriosclerosis, inhibitors of this kinase may also be useful for the treatment or prevention of various arteriosclerotic cardiovascular diseases, including angina pectoris, myocardial infarction, hypertensive vascular disease, stroke, heart failure, and arteriosclerosis obliterans. ROK has also been shown to be involved in endothelial contraction and enhancement of endothelial permeability which is thought to progress atherosclerosis.

The strategy of inhibiting ROK may also be useful for the treatment of other disorders associated with smooth muscle hyper-reactivity, such as bronchial asthma and glaucoma. Indeed, it has been recently demonstrated that ROK is involved in bronchial smooth muscle contraction and the regulation of aqueous humor outflow.

ROK is also thought to play a role in the negative regulation of bone marrow formation and that its inhibition may prove to be an appropriate new strategy for treatment of osteoporosis. Based upon rat model data, ROK inhibitors may also be useful for treatment of erectile dysfunction resulting from cavernosal smooth muscle relaxation. ROK inhibitors have also been implicated in treatment of AIDS through the proposed inhibition of HIV replication.

Inhibitors of this kinase have also been strongly implicated in the future treatment of cancer. It is known that constitutive activation of the Rho/ROK pathway contributes to the Ras transformation phenotype and mutations of Ras are thought to occur in as many as 25% of human tumours. Indeed pharmacological inhibition of ROK has been demonstrated to reduce both focus formation generated by Ras mutants and anchorage-independent growth in some colorectal cell lines. Evidence also exists to support a critical role for ROK in tumour cell invasion. To this end a ROK therapeutic has the potential for broad applicability to a wide range of cancer types.

In summary the early generation ROK inhibitors have shown promising efficacy in a variety of disease areas. The development of further ROK inhibitors with improved activity, selectivity and pharmacokinetic profiles is therefore needed to fully exploit the clinical potential of this target.

The invention addresses or ameliorates at least one of the disadvantages of the prior art, or provides a useful alternative.

Thus, in a first aspect the invention provides a compound selected from the specific group of compounds that comprises or consists of compounds of formula (I) or (II):

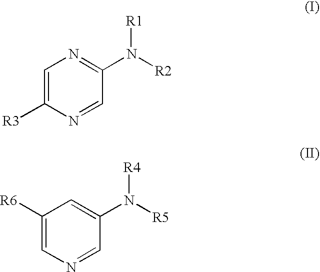

wherein:

R1 and R2 are joined to form a ring system, wherein the ring is preferably a 5 to 7 membered ring optionally substituted containing 1 to 3 heteroatoms selected from nitrogen and oxygen. More preferably, the ring is selected from 2-(2-hydroxy-ethyl)-piperidin-1-yl or 4-(2-hydroxy-ethyl)-piperazin-1-yl; 4-methyl-piperazin-1-yl; 4-pyridin-4-yl-piperazin-1-yl; 4-(2-dimethylamino-ethyl)-piperazin-1-yl; 4-(2-diethylamino-ethyl)-piperazin-1-yl; morpholin-4-yl; 4-(2-cyano-phenyl)-piperazin-1-yl; 4-methyl-[1,4]diazepan-1-yl; N-(2-dimethylamino-ethyl)-N-methyl-; 4-(3,4-dimethoxy-phenyl)-piperazin-1-yl; 4-pyridin-2-yl-piperazin-1-yl; 4-(2-hydroxy-ethyl)-piperazin-1-yl; 4-(furan-3-carbonyl)-piperazin-1-yl; 4-(2-pyrrolidin-1-yl-ethyl)-piperazin-1-yl; 2-(2-hydroxy-ethyl)-pyrrolidin-1-yl; or

R1 is H; and

R2 is 2-pyridin-4-yl-ethyl; 3-chloro-benzyl; benzo[1,3]dioxol-4-ylmethyl; 4-sulfonamide-benzyl; benzyl; thiophen-2-ylmethyl; 1-phenyl-ethyl; 4-(4-amino-benzoylamino)-phenyl; 4-methoxy-benzyl; 1-hydroxymethyl-2-methyl-propyl; 2-Pyridin-3-yl-ethyl; 4-phenoxy-phenyl; 4-fluoro-phenyl; 4-[ethyl-(2-hydroxy-ethyl)-amino]-phenyl; C1-C6 optionally substituted alkyl, preferably ethyl, propyl, 3-hydroxy-2,2-dimethyl-propyl, 3-hydroxy-propyl, 2-methoxy-ethyl, 2-hydroxy-ethyl, 2-hydroxymethyl-3-methyl-butyl, 1-hydroxymethyl-propyl, 2-morpholin-4-yl-ethyl, furan-2-yl-methyl; C3-C6 optionally substituted cycloalkyl preferably, cyclohexane; 5 to 7-membered optionally substituted containing 1 to 3 heteroatoms selected from nitrogen and oxygen, preferably piperazine ring, [1,4]diazepane or a pyrrolidine ring; R2 is optionally linked to the scaffold by a linker which includes 1 to 3 carbon atoms;

R3 is benzofuran-2-yl; naphthalen-2-yl; 3-4-methoxy-phenyl; 4-thiomethyl-phenyl; benzothiophen-2-yl; 4-pyridyl;. 4-methoxy-phenyl; quinolin-3-yl; benzo[1,3]dioxol-5-yl; 4-hydroxy-phenyl; 4-trifluoromethoxy-phenyl; 3-chloro-4-pyridyl; 3-4-5-methoxy-phenyl; 5-acetyl-thiophen-2-yl; 3-trifluoromethoxy-phenyl; 4-hydroxymethyl-phenyl; N-(4-Methoxy-phenyl)-benzamide-4-yl; 3-fluoro-4-chloro-phenyl; N-(2-Hydroxy-ethyl)-4-benzamide-4-yl; 3-hydroxy-phenyl; 3-acetylamino-phenyl; quinolin-7-yl; 2-methoxy-5-isopropyl-phenyl; 3-hydroxymethyl-phenyl; 3-pyridyl; hex-1-enyl; 4-cyano-phenyl; thiophen-3-yl; 3-nitro-phenyl; 3-chloro-phenyl; 2-methoxy-phenyl; 4-isopropyl-phenyl;

R4 and R5 are joined to form a ring system, wherein the ring is preferably 5 to 7 membered optionally substituted containing 1 to 3 heteroatoms selected from nitrogen and oxygen. More preferably, the ring is selected from 2-(2-hydroxy-ethyl)-piperidin-1-yl or 4-(2-hydroxy-ethyl)-piperazin-1-yl; 4-methyl-piperazin-1-yl; 4-pyridin-4-yl-piperazin-1-yl; 4-(2-dimethylamino-ethyl)-piperazin-1-yl; 4-(2-diethylamino-ethyl)-piperazin-1-yl; morpholin-4-yl; 4-(2-cyano-phenyl)-piperazin-1-yl; 4-methyl-[1,4]diazepan-1-yl; N-(2-dimethylamino-ethyl)-N-methyl-; 4-(3,4-dimethoxy-phenyl)-piperazin-1-yl; 4-pyridin-2-yl-piperazin-1-yl; 4-(2-hydroxy-ethyl)-piperazin-1-yl; 4-(furan-3-carbonyl)-piperazin-1-yl; 4-(2-pyrrolidin-1-yl-ethyl)-piperazin-1-yl; 2-(2-hydroxy-ethyl)-pyrrolidin-1-yl; or

R4 is H or Methyl; and

R5 is 3-hydroxy-phenyl; 3-hydroxybenzoyl; 4-bromo-benzyl; 4-methoxybenzyl; 2,5-hydroxybenzyl; 3-hydroxy-4-methoxy-benzyl; 3-chloro-benzyl; 3-fluoro-4-chloro-benzyl; 3-amino-benzyl; 3-trifluoromethoxy-benzyl; 4-hydroxy-benzyl; 4-amino-benzyl; 1H-Indol-6-yl; 3-hydroxy-benzyl; naphthalen-2-yl-methyl; benzo[1,3]dioxol-4-ylmethyl; 3,4-fluoro-benzyl; 3,4-chloro-benzyl; furan-3-yl-methyl; 4-methoxy-phenyl; 4-chloro-benzyl; 3-nitro-phenyl; 3,4-methoxy-phenyl; 3-bromo-phenyl; 4-chloro-phenyl; phenyl; 3-chloro-phenyl; 2-naphtyl; pyridin-3-yl-methyl; pyridin-4-yl-methyl; quinolin-3-yl-methyl; 4-isopropyl-phenyl; 4-chloro-benzyl; 3,4-methoxy-benzyl; 3-fluoro-4-chloro-phenyl; 4-trifluoromethoxy-phenyl; 4-cyano-phenyl; 4-metoxy benzyl, 4-methoxy-3-hydroxy benzyl; pyridin-4-yl-ethyl; piperidine-1-carboxylic acid benzyl ester 3-yl-methyl ; cyclohexane-methyl; 4-chlorobenzoyl; pyrrolidine-2-yl-methyl; C1-C6 optionally substituted alkyl, preferably ethyl, propyl, 3-hydroxy-2,2-dimethyl-propyl, 3-hydroxy-propyl, 2-methoxy-ethyl, 2-hydroxy-ethyl, 2-hydroxymethyl-3-methyl-butyl, 1-hydroxymethyl-propyl, 2-morpholin-4-yl-ethyl, furan-2-yl-methyl; C3-C8 optionally substituted cycloalkyl preferably cyclohexane; 5 to 7 membered optionally substituted containing 1 to 3 heteroatoms selected from nitrogen and oxygen, preferably piperazine ring, [1,4]diazepane or a pyrrolidine ring; R5 is optionally linked to the scaffold by a linker which includes 1 to 3 carbon atoms;

R6 is 3-carbamoyl-phenyl; 4-hydroxy-phenyl; 4-amino-phenyl; 3-amino-phenyl; phenyl; 1H-Indol-5-yl; 4-pyridyl; 3-hydoxy-phenyl; Benzo[1,3]dioxol-5-yl; 3-(2-Hydroxy-ethylcarbamoyl)-phenyl; 3-hydroxymethyl-phenyl; 3-acetylamino-phenyl; 4-hydroxymethyl-phenyl; 3-(2-dimethylamino-ethylcarbamoyl)-phenyl; thiophene-3-yl; 3-pyridyl; 3,4-methoxy-phenyl; 6-Bromo-1-carboxylic acid tert-butyl ester-indol-2-yl; 3-(2-hydroxy-ethylcarbamoyl)-phenyl; 3-Methanesulfonylamino-phenyl; 3-trifluoromethoxy-phenyl; 4-hydroxymethyl-phenyl; 4-methanesulfonyl-phenyl; quinolin-3-yl; 5-methoxy-pyridin-3-yl; 4-carbamoyl-phenyl; 4-acetylamino-phenyl; 4-Methylcarbamoyl-phenyl; 4-(2-Hydroxy-ethylcarbamoyl)-phenyl; quinolin-4-yl; quinolin-5-yl; isoquinolin-4-yl; 1H-pyrazol-4-yl; 3-chloro-pyridin-4-yl; 3-methoxy-pyridin-5-yl; 4-methoxy-pyridin-5-yl; 2-methyl-pyridin-4-yl; benzothiophene-2-yl; 3-chloro-pyridine-4-yl; 1H-pyrazol-3-yl; isoquinolin-3-yl; 4-carbamoyl-phenyl; 4-carbamoyl-phenyl; 3-(2-Hydroxy-ethylcarbamoyl)-phenyl; More preferably, R1 is hydrogen;

R2 is 2-pyridin-4-yl-ethyl; thiophen-2-ylmethyl; 4-sulfonamide-benzyl; or 3-chloro-benzyl;

R3 is benzothiophen-2-yl; naphthalen-2-yl; 3-4-methoxy-phenyl; or 4-pyridyl;

R4 is hydrogen;

R5 is 3-hydroxy-benzyl; 4-chloro-benzyl; naphthalen-2-yl-methyl; benzo[1,3]dioxol-4-ylmethyl; 3,4-fluoro-benzyl; 3,4-chloro-benzyl; or furan-3-yl-methyl; and

R6 is 3-carbamoyl-phenyl; 4-hydroxy-phenyl; or 4-pyridyl.

Most preferably a compound according to an embodiment of the invention has the structure of a compound of Table A or B below.

Any known compound having a structural formula identical to any one of the compounds covered by the formulae of scaffolds and permitted substitutions described herein is hereby explicitly disclaimed per se.

In a second aspect the invention provides a method for making a compound according to a first aspect of the invention, which method comprises at least one step or a series of consecutive steps from the scheme defined herein below.

In a third aspect the invention provides a group of at least two compounds comprising or consisting of a set of structurally related compounds having a core chemical structure (scaffold) of a general formula selected from the group consisting of formula I or II.

Preferably, an embodiment of a group of compounds according to the invention comprises compounds according to the first aspect of the invention, and said group of compounds has all or substantially all of the permitted substitutions represented by compounds therein.

In a fourth aspect the invention provides a method for making a group of compounds according to an aspect of the invention, which method comprises at least one step or a series of consecutive steps from the scheme defined herein below.

In a further aspect the invention provides an assay comprising a group of compounds, or one or more compounds according to the invention.

In a further aspect the invention provides use of an assay according to an embodiment of the invention for identifying a compound that has therapeutic affect.

In a further aspect the invention provides a pharmaceutical composition that comprises a compound according to an embodiment of the invention or a compound identified in an assay according to an embodiment of the invention.

In a further aspect the invention provides a compound according to an embodiment of the invention for use in therapy.

In a further aspect the invention provides use of a compound according to an embodiment of the invention in the manufacture of a medicament for treatment or prophylaxis of a condition characterised by abnormal kinase activity.

In a further aspect the invention provides use of a compound according to an embodiment of the invention in the manufacture of a medicament for treatment or prophylaxis of a condition selected from cardiovascular disease (coronary vasospasm, hypertensive disease, arteriosclerosis), stroke, cancer, erectile dysfunction, asthma, osteoporosis glaucoma and AIDS.

In a further aspect the invention provides a method of treatment of a condition characterised by abnormal kinase activity that comprises administering a pharmaceutically effective amount of a compound according to an embodiment of the invention.

In a further aspect the invention provides a method of treatment of a condition selected from cardiovascular disease (coronary vasospasm, hypertensive disease, arteriosclerosis), stroke, cancer, erectile dysfunction, asthma, osteoporosis glaucoma and AIDS that comprises administering a pharmaceutically effective amount of a compound according to an embodiment of the invention.

Compounds of the invention can be incorporated into pharmaceutical compositions suitable for administration. Such compositions typically comprise at least one compound of the invention and at least one pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound (e.g., a compound according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those-skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of-dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

The invention will now be described in detail with reference to specific examples of compounds and methods for their production.

Within this specification embodiments have been described in a way that enables a clear and concise specification to be written, but it will be appreciated that embodiments may be variously combined or separated without parting from the invention.

A compound according to an embodiment of the invention may be provided as a salt, preferably as a pharmaceutically acceptable salt of compounds of formula I or II. Examples of pharmaceutically acceptable salts of these compounds include those derived from organic acids such as acetic acid, malic acid, tartaric acid, citric acid, lactic acid, oxalic acid, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formation of salts of compounds for this invention include the hydroxides, carbonates, and bicarbonates of ammonia, lithium, sodium, calcium, potassium, aluminium, iron, magnesium zinc and the like. Salts can also be formed with suitable organic bases. Such bases suitable for the formation of pharmaceutically acceptable base addition salts with compounds of the present invention include organic bases which are nontoxic and strong enough to form salts. Such organic bases are already well known in the art and may include amino acids such as arginine and lysine, mono-, di-, or trihydroxyalkylamines such as mono-, di-, and triethanolamine, choline, mono-, di-, and trialkylamines, such as methylamine, dimethylamine, and trimethylamine, guanidine; N-methylglucosamine; N-methylpiperazine; morpholine; ethylenediamine; N-benzylphenethylamine; tris(hydroxymethyl) aminomethane; and the like.

Salts of compounds according to an embodiment of the invention may be prepared in a conventional manner using methods well known in the art. Acid addition salts of said basic compounds may be prepared by dissolving the free base compounds according to the first or second aspects of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g. by evaporation. The compounds of this invention may also exist in solvated or hydrated forms.

The invention also extends to prodrug of a compound according to an embodiment of the invention such as an ester or amide thereof. A prodrug is a compound that may be converted under physiological conditions or by solvolysis to a compound according to an embodiment of the invention or to a pharmaceutically acceptable salt of a compound according to an embodiment of the invention. A prodrug may be inactive when administered to a subject but is converted in vivo to an active compound of the invention.

A compound for use according to the invention may contain one or more asymmetric carbon atoms and may exist in racemic and optically active forms. A compound according to an embodiment of the invention may be in trans or cis form.

The invention will now be described in detail with reference to particular embodiments and examples of the invention.

Within the following table activity is presented as +, ++, or +++ representing active, more active and very active based on assays conducted at 1-100 μM.

TABLE A

| ROK | |
|---|---|
| activity | |
| ++ | [5-(3,4-Dimethoxy-phenyl)-pyrazin-2-yl]-(2-pyridin-4-yl- |
| | ethyl)-amine |
| + | 4-[5-(2-Pyridin-4-yl-ethylamino)-pyrazin-2-yl]-phenol |
| ++ | (2-Pyridin-4-yl-ethyl)-[5-(4-trifluoromethoxy-phenyl)-pyrazin- |
| | 2-yl]-amine |
| ++ | (2-Pyridin-4-yl-ethyl)-[5-(3,4,5-trimethoxy-phenyl)-pyrazin-2- |
| | yl]-amine |
| ++ | (2-Pyridin-4-yl-ethyl)-[5-(3-trifluoromethoxy-phenyl)-pyrazin- |
| | 2-yl]-amine |
| ++ | (5-Benzo[1,3]dioxol-5-yl-pyrazin-2-yl)-(2-pyridin-4-yl-ethyl)- |
| | amine |
| ++ | 4-(5-Benzylamino-pyrazin-2-yl)-phenol |
| + | N-(2-Hydroxy-ethyl)-3-[5-(2-pyridin-4-yl-ethylamino)-pyrazin- |
| | 2-yl]-benzamide |
| ++ | N-(4-Methoxy-phenyl)-4-[5-(2-pyridin-4-yl-ethylamino)- |
| | pyrazin-2-yl]-benzamide |
| ++ | [5-(4-Methoxy-phenyl)-pyrazin-2-yl]-(2-pyridin-4-yl-ethyl)- |
| | amine |
| + | {4-[5-(2-Pyridin-4-yl-ethylamino)-pyrazin-2-yl]-phenyl}- |
| | methanol |
| ++ | (5-Naphthalen-2-yl-pyrazin-2-yl)-(2-pyridin-4-yl-ethyl)-amine |
| ++ | Benzo[1,3]dioxol-5-ylmethyl-(5-pyridin-4-yl-pyrazin-2-yl)- |
| | amine |
| + | (4-Methoxy-benzyl)-(5-pyridin-4-yl-pyrazin-2-yl)-amine |
| + | [5-(3-Chloro-4-fluoro-phenyl)-pyrazin-2-yl]-(2-pyridin-4-yl- |
| | ethyl)-amine |
| + | 1-{5-[5-(2-Pyridin-4-yl-ethylamino)-pyrazin-2-yl]-thiophen-2- |
| | yl}-ethanone |
| ++ | (5-Benzofuran-2-yl-pyrazin-2-yl)-(2-pyridin-4-yl-ethyl)-amine |
| ++ | (5-Benzo[b]thiophen-2-yl-pyrazin-2-yl)-(2-pyridin-4-yl-ethyl)- |
| | amine |
| ++ | [5-(4-Methylsulfanyl-phenyl)-pyrazin-2-yl]-(2-pyridin-4-yl- |
| | ethyl)-amine |
| ++ | (2-Pyridin-4-yl-ethyl)-(5-quinolin-3-yl-pyrazin-2-yl)-amine |
| ++ | (5-Pyridin-4-yl-pyrazin-2-yl)-thiophen-2-ylmethyl-amine |
| + | 3-Methyl-2-(5-pyridin-4-yl-pyrazin-2-ylamino)-butan-1-ol |
| + | 2-[1-(5-Pyridin-4-yl-pyrazin-2-yl)-piperidin-2-yl]-ethanol |
| + | 4-[5-(3-Chloro-benzylamino)-pyrazin-2-yl]-phenol |
| ++ | (3-Chloro-benzyl)-(5-pyridin-4-yl-pyrazin-2-yl)-amine |
| ++ | 4-[5-(1-Phenyl-ethylamino)-pyrazin-2-yl]-phenol |
| + | (5-Naphthalen-2-yl-pyrazin-2-yl)-(2-pyridin-3-yl-ethyl)-amine |
| ++ | 4-Amino-N-{4-[5-(3-chloro-pyridin-4-yl)-pyrazin-2-ylamino]- |
| | phenyl}-benzamide |
| ++ | 4-[(5-Pyridin-4-yl-pyrazin-2-ylamino)-methyl]- |
| | benzenesulfonamide |
| ++ | [3,4′]Bipyridinyl-5-yl-naphthalen-2-ylmethyl-amine |
| + | 4-{5-[(Naphthalen-2-ylmethyl)-amino]-pyridin-3-yl}-phenol |
| ++ | 3-[5-(3,4-Dichloro-benzylamino)-pyridin-3-yl]-benzamide |
| ++ | [3,4′]Bipyridinyl-5-yl-(3,4-dichloro-benzyl)-amine |
| + | 4-[5-(3,4-Dichloro-benzylamino)-pyridin-3-yl]-phenol |
| ++ | 3-[5-(4-Chloro-benzylamino)-pyridin-3-yl]-benzamide |
| +++ | [3,4′]Bipyridinyl-5-yl-(4-chloro-benzyl)-amine |
| ++ | 4-[5-(4-Chloro-benzylamino)-pyridin-3-yl]-phenol |
| ++ | 3-[5-(3,4-Difluoro-benzylamino)-pyridin-3-yl]-benzamide |
| ++ | [3,4′]Bipyridinyl-5-yl-(3,4-difluoro-benzyl)-amine |
| + | 4-[5-(3,4-Dimethoxy-benzylamino)-pyridin-3-yl]-phenol |
| + | 3-{5-[(Benzo[1,3]dioxol-5-ylmethyl)-amino]-pyridin-3-yl}- |
| | benzamide |
| ++ | Benzo[1,3]dioxol-5-ylmethyl-[3,4′]bipyridinyl-5-yl-amine |
| ++ | 4-{5-[(Benzo[1,3]dioxol-5-ylmethyl)-amino]-pyridin-3-yl}- |
| | phenol |
| ++ | 4-[5-(3,4-Difluoro-benzylamino)-pyridin-3-yl]-phenol |
| ++ | 3-[5-(3-Hydroxy-benzylamino)-pyridin-3-yl]-N-(2-hydroxy- |
| | ethyl)-benzamide |
| +++ | 3-[5-(3-Hydroxy-benzylamino)-pyridin-3-yl]-benzamide |
| ++ | 3-[(5-Benzo[1,3]dioxol-5-yl-pyridin-3-ylamino)-methyl]-phenol |
| ++ | 3-([3,4′]Bipyridinyl-5-ylaminomethyl)-phenol |
| + | 3-{[5-(4-Hydroxymethyl-phenyl)-pyridin-3-ylamino]-methyl}- |
| | phenol |
| + | N-{3-[5-(3-Hydroxy-benzylamino)-pyridin-3-yl]-phenyl}- |
| | acetamide |
| +++ | (5-(4′-hydroxy-phenyl)-pyridin-3-yl)-(3-hydroxy-benzyl)-amine |
| + | 3-{[5-(3-Hydroxymethyl-phenyl)-pyridin-3-ylamino]-methyl}- |
| | phenol |
| + | (5-(3′-hydroxy-phenyl)-pyridin-3-yl)-(3-hydroxy-benzyl)-amine |
| + | 3-{5-[(Furan-3-ylmethyl)-amino]-pyridin-3-yl}-benzamide |
| ++ | [3,4′]Bipyridinyl-5-yl-furan-3-ylmethyl-amine |
| ++ | 4-{5-[(Furan-3-ylmethyl)-amino]-pyridin-3-yl}-phenol |
| + | 3-{5-[(Pyridin-3-ylmethyl)-amino]-pyridin-3-yl}-benzamide |
| ++ | [3,4′]Bipyridinyl-5-yl-(3-chloro-phenyl)-amine |
| + | 3-[5-(3-Bromo-phenylamino)-pyridin3-yl]-benzamide |
| ++ | [3,4′]Bipyridinyl-5-yl-(3-bromo-phenyl)-amine |
| ++ | [3,4′]Bipyridinyl-5-yl-(3-nitro-phenyl)-amine |
| + | 3-[5-(4-Methoxy-phenylamino)-pyridin-3-yl]-benzamide |
| ++ | 3-[5-(4-Methoxy-phenylamino)-pyridin-3-yl]-phenol |
| + | N-(2-Dimethylamino-ethyl)-3-[5-(naphthalen-2-ylamino)- |
| | pyridin-3-yl]-benzamide |
| + | 3-[5-(Naphthalen-2-ylamino)-pyridin-3-yl]-phenol |
| + | 3-[5-(4-Chloro-phenylamino)-pyridin-3-yl]-phenol |
| +++ | 3-[(3′-Chloro-[3,4′]bipyridinyl-5-ylamino)-methyl]-phenol |
| +++ | 3-[(5-Quinolin-5-yl-pyridin-3-ylamino)-methyl]-phenol |
| ++ | 3-{[5-(1H-Pyrazol-3-yl)-pyridin-3-ylamino]-methyl}-phenol |
| + | 3-[(5-Isoquinolin-4-yl-pyridin-3-ylamino)-methyl]-phenol |
| + | N-{4-[5-(3-Hydroxy-benzylamino)-pyridin-3-yl]-phenyl}- |
| | acetamide |
| +++ | 4-[5-(3-Hydroxy-benzylamino)-pyridin-3-yl]-benzamide |
| + | 3-{[5-(4-Amino-phenyl)-pyridin-3-ylamino]-methyl}-phenol |
| + | 3-{[5-(1H-Indol-5-yl)-pyridin-3-ylamino]-methyl}-phenol |
| + | 3-[(5′-Methoxy-[3,3′]bipyridinyl-5-ylamino)-methyl]-phenol |
| ++ | 3-[5-(3-Hydroxy-benzylamino)-pyridin-3-yl]-N-(2-hydroxy- |
| | ethyl)-benzamide |
| + | 3-[(5-Phenyl-pyridin-3-ylamino)-methyl]-phenol |
| + | 3-{[5-(3-Amino-phenyl)-pyridin-3-ylamino]-methyl}-phenol |
| + | 4-{5-[(3-hydroxy-benzyl)-methyl-amino]-pyridin-3-yl}-phenol |
| ++ | 5-{[5-(4-Hydroxy-phenyl)-pyridin-3-ylamino]-methyl}- |
| | benzene-1,3-diol |
| ++ | 3-[5-(3,5-Dihydroxy-benzylamino)-pyridin-3-yl]-benzamide |
| +++ | [3,4′]Bipyridinyl-5-yl-(4-bromo-benzyl)-amine |
| ++ | [3,4′]Bipyridinyl-5-yl-(3-chloro-benzyl)-amine |
| ++ | 4-[5-(3-Chloro-benzylamino)-pyridin-3-yl]-phenol |
| ++ | 3-[5-(3-Chloro-benzylamino)-pyridin-3-yl]-benzamide |
| +++ | [3,4′]Bipyridinyl-5-yl-(4-methoxy-benzyl)-amine |
| ++ | 4-[5-(4-Methoxy-benzylamino)-pyridin-3-yl]-phenol |
| + | 3-[5-(4-Methoxy-benzylamino)-pyridin-3-yl]-benzamide |
| ++ | 3-[5-(4-Hydroxy-benzylamino)-pyridin-3-yl]-benzamide |
| ++ | 5-{[5-(4-Hydroxy-phenyl)-pyridin-3-ylamino]-methyl}-2- |
| | methoxy-phenol |
| ++ | 3-[5-(3-Hydroxy-4-methoxy-benzylamino)-pyridin-3-yl]- |
| | benzamide |
| ++ | 3-([3,4′]Bipyridinyl-5-ylamino)-phenol |
| ++ | 4-[5-(3-Hydroxy-phenylamino)-pyridin-3-yl]-phenol |
| ++ | 3-[5-(3-Hydroxy-phenylamino)-pyridin-3-yl]-benzamide |
| ++ | [3,4′]Bipyridinyl-5-yl-cyclohexylmethyl-amine |
| + | 4-[5-(Cyclohexylmethyl-amino)-pyridin-3-yl]-phenol |
| + | 3-[5-(Cyclohexylmethyl-amino)-pyridin-3-yl]-benzamide |
| +++ | [3,4′]Bipyridinyl-5-yl-(4-chloro-3-fluoro-benzyl)-amine |
| ++ | 3-[5-(4-Chloro-3-fluoro-benzylamino)-pyridin-3-yl]-benzamide |
| ++ | [3,4′]Bipyridinyl-5-yl-(3-trifluoromethoxy-benzyl)-amine |
| + | 4-[5-(3-Trifluoromethoxy-benzylamino)-pyridin-3-yl]-phenol |
| ++ | 3-[5-(3-Trifluoromethoxy-benzylamino)-pyridin-3-yl]- |
| | benzamide |
| + | [3,4′]Bipyridinyl-5-yl-pyrrolidin-2-ylmethyl-amine |
| ++ | 4-{5-[(Pyrrolidin-2-ylmethyl)-amino]-pyridin-3-yl}-phenol |
| + | 3-{5-[(Pyrrolidin-2-ylmethyl)-amino]-pyridin-3-yl}-benzamide |

An embodiment of a compound according to the invention can be produced according to the following scheme.

General Scheme for Synthesising Compounds of Formula (I)

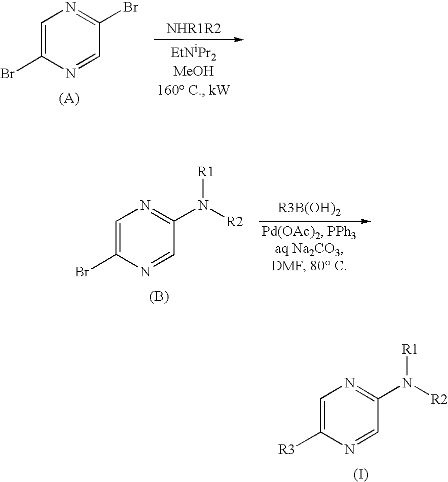

2,5-Dibromopyrazine (A) can be aminated with amines. The resultant compounds (B) can then be reacted with the boronic acids to yield the final compounds of formula (I).

General Procedures:

Typical example of compound of formula (B), as described in the general reaction scheme; (5-Bromo-pyrazin-2-yl)-(2-pyridin-4-yl-ethyl)-amine

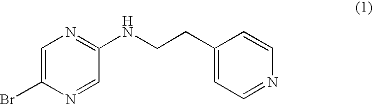

To a solution of (A) in 3ml of n-butanol was added 0.61 g of 2-pyridine-4-yl-ethylamine and 0.65 g of Hunigs base. The mixture was heated at 150° C. in a microwave for 0.5 hr. The mixture was poured into dichioromethane. The organic phase was washed with water and brine before being dried on magnesium sulfate. The mixture was filtrate and concentrated in vacuo. The crude product was purified by flash chromatography on silica gel using petroleum ether and ethyl acetate as a eluent.

¹H (270 MHz, CDCl₃) 2.91-2.96. (2H, t, J 6.8), 3.62-3.70 (2H, m), 7.14-7.16 (2H, m), 7.64-7.65 (1H, d, J 1.2), 8.10-8.11 (1H, d, J 1.2), 8.52-8.55 (2H, m); HPLC 82%; m/z (ES) 279 [M+H]⁺.

General Procedure for the Synthesis of Compounds of Formula (I).

To a solution of the required intermediates (B) in DMF (0.3 mmol, 0.5 ml) was added a solution of boronic acid in DMF (0.36 mmol, 0.6 ml) and 1.5M Na₂CO₃(aq.) solution (0.75 mmol, 0.5 ml) under nitrogen. Two solutions of palladium acetate (95 mg) and triphenylphosphine (335 mg) in 1,4-dioxane (15 ml) were freshly prepared and placed in a sonication bath for 2 min. The palladium catalyst (0.3 ml) was added. The reaction was then heated at 80° C. with agitation for 16 h under nitrogen. The reaction mixtures were filtered and purified by preparative reverse phase HPLC or on silicagel by flash chromatography.

Typical example of compound of formula (I), (5-Benzo[b]thiophen-2-yl-pyrazin-2-yl)-(2-pyridin-4-yl-ethyl)-amine

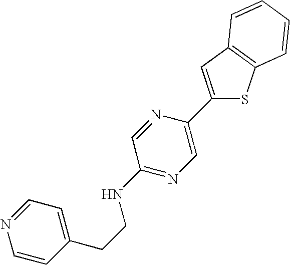

A mixture of 138 mg of benzothiophene-2-boronuc acid, 130 mg of (5-Bromo-pyrazin-2-yl)-(2-pyridin-4-yl-ethyl)-amine, 22 mg of palladium acetate and 37 mg of triphenylphosphine in 3 ml of DMF and 1.5 ml and 2M aqueous sodium carbonate solution was prepared at room temperature. The stirred mixture was heated at 100° C. in a Discovery microwave CEM for 0.5 hr. The reaction mixture was filtered through celite, then diluted with water, extracted with ether. The ether extracts were dried over magnesium sulfate, then concentrated to dryness. The crude product was purified by flash chromatography using petroleum ether and ethyl actetate as a eluent.

¹H (270 MHz, CDCl₃) 2.3(2H, t), 3.7 (2H, q), 4.95 (1H, bs), 7.15 (1H,s), 7.35 (2H, m), 7.45 (1H, m), 7.65 (1H, m), 7.75 (1H, m), 7.85(1H, m), 7.9 (1H, s), 8.55 (3H, m); HPLC 96.8%; m/z (ES) 333 [M+H]⁺.

Scheme for Synthesising Compounds of Formula (II)-(IIa and IIb)

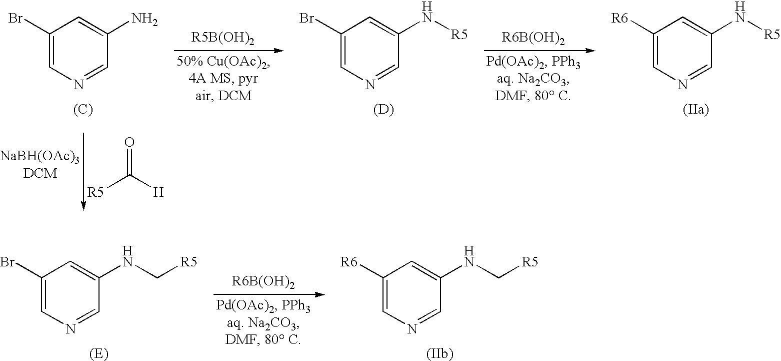

3-Amino-5-bromopyridine (C) can be subjected to a copper mediated N-arylation with boronic acids, and the resultant compounds (D) then subjected to Suzuki cross coupling reaction using further boronic acids to yield final compounds of formula (IIa). Alternatively, compounds with the general structure (E) can be synthesised through a reductive amination. Functionalisation at C5 with the boronic acids yields final compounds with the general formula (IIb).

General Procedures:

Typical example of compound of formula (D), as described in the general reaction scheme; (5-bromo-pyridin-3-yl)-(3-nitro-phenyl)-amine.

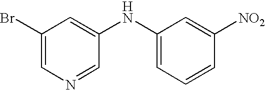

3-Amino-5-bromopyridine (3.11 g, 18 mmol), 3-nitrophenylboronic acid (6.28 g, 36 mmol), copper(II) acetate (1.63 g, 9 mmol), 4 Å molecular sieves (3 g) and pyridine (2.9 ml, 36 mmol) in DCM (50 ml) was stirred vigorously in an open top vessel for 18 h. The reaction was filtered and the residue was washed with methanol. SiO₂ (10 g) was added to the solution and concentrated in vacuo to dryness. The resultant solid was chromatographed (SiO₂, 20%-50% EtOAc in hexane) to afford the desired product as a bright yellow solid. ¹H (270 MHz, CDCl₃) 6.08(1H, br.s, NH), 7.39-7.40(1H, m, r), 7.45-7.60(1H, m, Ar), 7.62(1H, s, Ar), 7.83-7.89(2H, m, Ar), 8.34-8.37 (2H, m, Ar); HPLC: Rₜ 2.06 (77.89%); m/z (ES): 294(100%, M⁺).

Typical example of compound of formula (D), as described in the general reaction scheme; (5-bromo-pyridin-3-yl)-phenyl-amine.

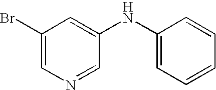

¹H (250 MHz, CDCl₃) 5.87 (1H, br.s), 7.07-7.53 (6H, m), 8.16-8.24 (2H, m); HPLC 92%; m/z (ES) 249 [M+H]⁺.

Typical example of compound of formula (D), as described in the general reaction scheme; (5-bromo-pyridin-3-yl)-(4-methoxy-phenyl)-amine.

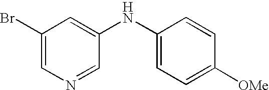

¹H (250 MHz, CDCl₃) 3.82 (3H, s) 5.61 (1H, br.s), 6.89-6.92 (2H, d, J 8.9) 7.07-7.11 (2H, d, J 8.9), 7.27-7.29 (1H, m) 8.06-8.11 (2H, m); HPLC 100%; m/z (ES) 279 [M+H]⁺.

Typical example of compound of formula (D), as described in the general reaction scheme; (5-bromo-pyridin-3-yl)-(4-chloro-phenyl)-amine.

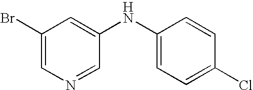

¹H (250 MHz, CDCl₃) 6.11 (1H, br.s), 7.02-7.06 (2H, d, J 8.8) 7.27-7.31 (2H, d, J 8.8), 7.48-7.50 (1H, m) 8.18-8.24 (2H, m); HPLC 90%; m/z (ES) 283 [M+H]⁺.

General Procedure for the Synthesis of Compounds of Formula (IIa).

To a solution of the required intermediates (D) in DMF (0.3 mmol, 0.5 ml) was added under nitrogen a solution of boronic acid in DMF (0.36 mmol, 0.6 ml) and 1.5M Na₂CO₃(aq.) solution (0.75 mmol, 0.5 ml). Two solutions of palladium acetate (95 mg) and triphenylphosphine (335 mg) in 1,4-dioxane (15 ml) were freshly prepared and placed in a sonication bath for 2 min. The palladium catalyst (0.3 ml) was then added to reaction mixture under nitrogen. The reaction mixture was heated at 80° C. with agitation for 16 h. The reaction mixtures were filtered and purified by preparative reverse phase HPLC or purified on silicagel by flash chromatography.

Typical example of compound of formula (IIa), as described in the general reaction scheme; [5-(4-methanesulphoylphenyl)pyridin-3-yl]-phenylamine

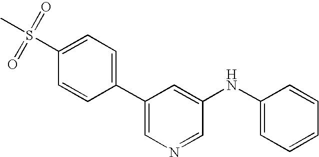

¹H (250 MHz, CDCl₃) 3.86 (3H, s), 6.23-6.43 (6H, m) 7.63-7.71 (3H, m), 7.95-7.96 (2H, m), 8.33-8.34 (2H, m); HPLC 100%; m/z (ES) 325 [M+H]⁺.

Typical example of compound of formula (IIa), as described in the general reaction scheme; phenyl-(5-quinolin-3-yl-pyridin-3-yl)amine.

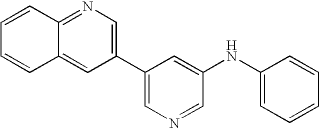

¹H (250 MHz, CDCl₃) 7.16-7.24 (3H, m), 7.38-7.44 (2H, m) 7.71-7.77 (1H, m), 7.7.86-7.93 (1H, m), 7.99-8.04 (2H, m), 8.24-8.27 (1H, m),8.44-8.45 (1H, m), 8.53-8.56 (2H, m), 9.23-9.24 (1H, m); HPLC 98%; m/z (ES) 298 [M+H]⁺.

Typical example of compound of formula (E), as described in the general reaction scheme; (5-bromo-pyridin-3-yl) -(4-chloro-benzyl)-amine.

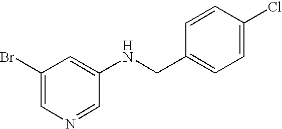

3-Amino-5-bromopyridine (2.04 g, 13 mmol), 4-chlorobenzaldehyde (1.83 g, 13 mmol) and sodium triacetoxyborohydride (3.86 g, 18.2 mmol) in DCM (40 ml) was stirred at room temperature for 16 h. The reaction was taken up in DCM. The solution was washed with water and brine. The organic layer was dried over MgSO₄ and concentrated in vacuo. The resultant solid was recrystallised from hexane/DCM to afford the desired product as an off-white solid. ¹H (270 MHz, CDCl₃) 4.24-4.68(3H, m, NH, CH₂), 6.98-7.00(1H, m, Ar), 7.25-7.36(4H, m, Ar), 7.95-8.02(2H, m, Ar); HPLC: Rₜ 1.94 (98.70%); m/z (ES): 297(100%, M⁺).

Typical example of compound of formula (E), as described in the general reaction scheme; (5-bromo-pyridin-3-yl)-furan-3-ylmethyl-amine.

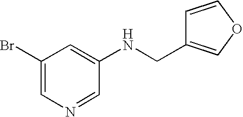

¹H (250 MHz, CDCl₃) 4.12-4.16 (3H, m), 6.39-6.40 (1H, m) 7.04-7.06 (1H, m), 7.42-7.43 (2H, m) 7.95-8.01 (2H, m); HPLC 980/o; m/z (ES) 253 [M+H]⁺.

Typical example of compound of formula (E), as described in the general reaction scheme; (5-bromo-pyridin-3-yl)-(3,4-difluoro-benzyl)-amine.

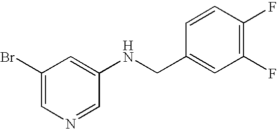

¹H (250 MHz, CDCl₃) 4.30-4.44 (3H, m), 6.97-7.20 (4H, m) 7.95-8.01 (2H, m); HPLC 95%; m/z (ES) 299 [M+H]⁺.

Typical example of compound of formula (E), as described in the general reaction scheme; (5-bromo-pyridin-3-yl)-(3,4-dichloro-benzyl)-amine.

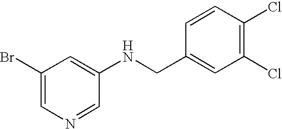

¹H (250 MHz, CDCl₃) 4.30-4.42 (3H, m), 6.97-6.98 (1H, m), 7.13-7.20 (1H, m), 7.39-7.47 (2H, m), 7.92-8.01 (2H, m); HPLC 75%; m/z (ES) 331 [M+H]⁺.

Typical example of compound of formula (E), as described in the general reaction scheme; (5-bromo-pyridin-3-yl)-(3-hydroxy-benzyl)-amine.

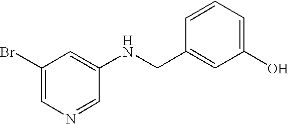

¹H (250 MHz, CDCl₃) 9.35 (1H, s), 7.84 (1H, s), 7.68 (1H, s), 7.04-7.00 (1H, m), 6.96-6.94 (1H, m), 6.74-6.71 (1H, m), 6.66-6.32 (2H, m), 6.54-6.52 (1H, m), 4.12-4.11 (2H, m); LCMS 97%; m/z (CI) [M+H]⁺279.

General Procedure for the Synthesis of Compounds of the Formula (IIb)

To a solution of the required intermediates. (E) in DMF (0.3 mmol, 0.5 ml) was added a solution of boronic acid in DMF (0.36 mmol, 0.6 ml) and 1.5M Na₂CO₃(aq.) solution (0.75 mmol, 0.5 ml). The reaction was performed under nitrogen. Two solutions of palladium acetate (95 mg) and triphenylphosphine (335 mg) in 1,4-dioxane (15 ml) were freshly prepared and placed in a sonication bath for 2 min. The palladium catalyst (0.3 ml) was added to reaction mixture under nitrogen. The reaction mixture was heated at 80° C. with agitation for 16 h. The reaction mixtures were filtered and purified by preparative reverse phase HPLC or purified by flash chromatography on silicagel.

Typical example of compound of formula (IIb), as described in the general reaction scheme; (5′-methoxy-[3,3′]bipyridinyl-5-yl)-naphthalen-2-ylmethyl-amine.

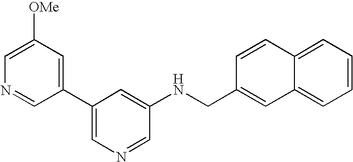

¹H (250 MHz, CDCl₃) 3.83(3H, s), 4.66 (2H, s), 7.24-7.26 (1H, m) 7.40-7.53 (4H, m), 7.79-7.89 (4H, m), 8.11-8.12 (1H, m), 8.34-8.42 (3H, m); HPLC 100%; m/z (ES) 342 [M+H]⁺.

Typical example of compound of formula (IIb), as described in the general reaction scheme; naphthalen-2-ylmethyl-(5-pyrimidin-5-yl-pyridin-3-yl)-amine.

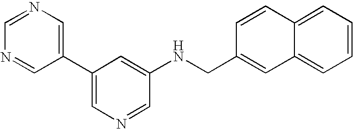

¹H (250 MHz, CDCl₃) 4.72 (2H, s), 7.13-7.15 (1H, m) 7.62-7.68 (1H, m), 7.78-7.90 (2H, m), 8.20-8.22 (2H, m), 8.30-8.31 (2H, m),8.89 (2H, s), 9.08-9.09 (1H, m), 9.25 (1H, s); HPLC 100%; m/z (ES) 314 [M+H]⁺.

Typical example of compound of formula (IIb), as described in the general reaction scheme; (5-(4′-hydroxy-phenyl)-pyridin-3-yl)-(3-hydroxy-benzyl)-amine

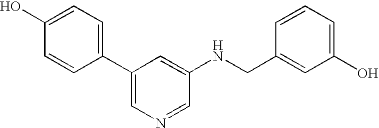

¹H (250 MHz, CDCl₃) 9.46 (1H, s), 9.20 (1H, s), 7.83 (1H, s), 7.76-7.75 (1H, m), 7.28-7.26 (2H, m), 7.00-6.97 (1H, m), 6.88-6.87 (1H, m), 6.71-6.65 (4H, m), 6.50-6.48 (1H, m), 6.40-6.37 (1H, m), 4.16-4.14 (2H, m); LCMS 97%; m/z (ES) [M+H]⁺293.

Typical example of compound of formula (IIb), as described in the general reaction scheme; 3-[5-(3-Hydroxy-benzylamino)-pyridin-3-yl]-benzamide

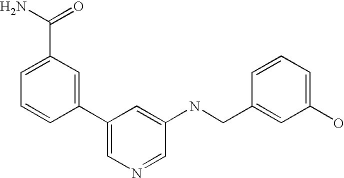

¹H (250 MHz, CDCl₃) 9.23 (1H, s), 7.99 (3H, s), 7.89-7.88 (1H, m), 7.78-7.57 (1H, m), 7.64-7.62 (1H, m), 7.45-7.42 (1H, m), 7.35 (1H, s), 7.07-7.01 (2H, m), 6.72-6.68 (2H, m), 6.53-6.50 (2H, m), 4.22-4.20 (2H, m); LCMS 99%; m/z (CI) [M+H]⁺320.

Typical example of compound of formula (IIb), as described in the general reaction scheme [3,4′]Bipyridinyl-5-yl-(4-chloro-benzyl)-amine

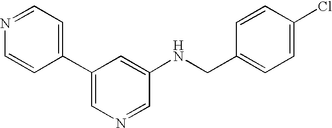

¹H (250 MHz, CDCl₃) 8.80-8.78 (2H, m), 8.32 (1H, s), 8.22-8.21 (1H, m), 7.81-7.79 (2H, m), 7.60-7.55 (4H, m), 7.38-7.37 (1H, m), 6.95-6.92 (1H, m), 4.57-4.56 (2H, m); LCMS 100%; m/z (ES) [M+H]⁺296.

Other examples of compounds of general formula (I), (IIa) or (IIb) prepared by the above procedures are recorded in Table B. Compounds were characterised by mass spectrometry using single quadrupole instrumentation with an electrospray source.

TABLE B

| | Mol. | | | | |
|---|---|---|---|---|---|
| Compound | Weight | MS data | M + 1 | M + Acetonitrile + 1 | 2M + 1 |
| [5-(3,4-Dimethoxy-phenyl)- | 336.4 | M + 1, | 337 | 378 | 673 |
| pyrazin-2-yl]-(2-pyridin-4-yl- | | M + Acetonitrile + 1, | | | |
| ethyl)-amine | | 2M + 1 | | | |
| 4-[5-(2-Pyridin-4-yl- | 292.3 | M + 1, | 293 | 334 | 585 |
| ethylamino)-pyrazin-2-yl]- | | M + Acetonitrile + 1, | | | |
| phenol | | 2M + 1 | | | |
| (2-Pyridin-4-yl-ethyl)-[5-(4- | 360.3 | M + 1, | 361 | 402 | 721 |
| trifluoromethoxy-phenyl)- | | M + Acetonitrile + 1, | | | |
| pyrazin-2-yl]-amine | | 2M + 1 | | | |
| (2-Pyridin-4-yl-ethyl)-[5- | 366.4 | M + 1, | 367 | 408 | 733 |
| (3,4,5-trimethoxy-phenyl)- | | M + Acetonitrile + 1, | | | |
| pyrazin-2-yl]-amine | | 2M + 1 | | | |
| (2-Pyridin-4-yl-ethyl)-[5-(3- | 360.3 | M + 1, | 361 | 402 | 721 |
| trifluoromethoxy-phenyl)- | | M + Acetonitrile + 1, | | | |
| pyrazin-2-yl]-amine | | 2M + 1 | | | |
| (5-Benzo[1,3]dioxol-5-yl- | 320.4 | M + 1, | 321 | 362 | 641 |
| pyrazin-2-yl)-(2-pyridin-4-yl- | | M + Acetonitrile + 1, | | | |
| ethyl)-amine | | 2M + 1 | | | |
| 4-(5-Benzylamino-pyrazin-2- | 277.3 | M + 1, | 278 | 319 | 555 |
| yl)-phenol | | M + Acetonitrile + 1, | | | |
| | | 2M + 1 | | | |
| N-(2-Hydroxy-ethyl)-3-[5-(2- | 363.4 | M + 1, 2M + 1 | 364 | | 727 |
| pyridin-4-yl-ethylamino)- | | | | | |
| pyrazin-2-yl]-benzamide | | | | | |
| N-(4-Methoxy-phenyl)-4-[5- | 425.5 | M + 1, | 426 | 467 | |
| (2-pyridin-4-yl-ethylamino)- | | M + Acetonitrile + 1 | | | |
| pyrazin-2-yl]-benzamide | | | | | |
| [5-(4-Methoxy-phenyl)- | 306.4 | M + 1, | 307 | 348 | 613 |
| pyrazin-2-yl]-(2-pyridin-4- | | M + Acetonitrile + 1, | | | |
| yl-ethyl)-amine | | 2M + 1 | | | |
| {4-[5-(2-Pyridin-4-yl- | 306.4 | M + 1, | 307 | 348 | 613 |
| ethylamino)-pyrazin-2-yl]- | | M + Acetonitrile + 1, | | | |
| phenyl}-methanol | | 2M + 1 | | | |
| (5-Naphthalen-2-yl-pyrazin- | 326.4 | M + 1, | 327 | 368 | 653 |
| 2-yl)-(2-pyridin-4-yl-ethyl)- | | M + Acetonitrile + 1, | | | |
| amine | | 2M + 1 | | | |
| Benzo[1,3]dioxol-5-ylmethyl- | 306.3 | M + 1 | 307 | | |
| (5-pyridin-4-yl-pyrazin-2-yl)- | | | | | |
| amine | | | | | |
| (4-Methoxy-benzyl)-(5- | 292.3 | M + 1 | 293 | | |
| pyridin-4-yl-pyrazin-2-yl)- | | | | | |
| amine | | | | | |
| [5-(3-Chloro-4-fluoro- | 328.8 | M + 1, | 329 | 370 | 657 |
| phenyl)-pyrazin-2-yl]-(2- | | M + Acetonitrile + 1, | | | |
| pyridin-4-yl-ethyl)-amine | | 2M + 1 | | | |
| 1-{5-[5-(2-Pyridin-4-yl- | 324.4 | M + 1, | 325 | 366 | 649 |
| ethylamino)-pyrazin-2-yl]- | | M + Acetonitrile + 1, | | | |
| thiophen-2-yl}-ethanone | | 2M + 1 | | | |
| (5-Benzofuran-2-yl-pyrazin- | 316.4 | M + 1, | 317 | 358 | 633 |
| 2-yl)-(2-pyridin-4-yl-ethyl)- | | M + Acetonitrile + 1, | | | |
| amine | | 2M + 1 | | | |
| (5-Benzo[b]thiophen-2-yl- | 332.4 | M + 1, | 333 | 374 | 665 |
| pyrazin-2-yl)-(2-pyridin-4-yl- | | M + Acetonitrile + 1, | | | |
| ethyl)-amine | | 2M + 1 | | | |
| [5-(4-Methylsulfanyl-phenyl)- | 322.4 | M + 1, | 323 | 364 | 645 |
| pyrazin-2-yl]-(2-pyridin-4-yl- | | M + Acetonitrile + 1, | | | |
| ethyl)-amine | | 2M + 1 | | | |
| (2-Pyridin-4-yl-ethyl)-(5- | 327.4 | M + 1 | 328 | | |
| quinolin-3-yl-pyrazin-2-yl)- | | | | | |
| amine | | | | | |
| (5-Pyridin-4-yl-pyrazin-2-yl)- | 268.3 | M + 1 | 269 | | |
| thiophen-2-ylmethyl-amine | | | | | |
| 3-Methyl-2-(5-pyridin-4-yl- | 258.3 | M + 1 | 259 | | |
| pyrazin-2-ylamino)-butan-1- | | | | | |
| ol | | | | | |
| 2-[1-(5-Pyridin-4-yl-pyrazin- | 284.4 | M + 1 | 285 | | |
| 2-yl)-piperidin-2-yl]-ethanol | | | | | |
| 4-[5-(3-Chloro-benzylamino)- | 311.8 | M + 1, | 312 | | |
| pyrazin-2-yl]-phenol | | Triphenylphosphine | | | |
| | | oxide + 1 | | | |
| (3-Chloro-benzyl)-(5-pyridin- | 296.8 | M + 1 | 297 | | |
| 4-yl-pyrazin-2-yl)-amine | | | | | |
| 4-[5-(1-Phenyl-ethylamino)- | 291.4 | M + 1, 2M + 1 | 292 | | 583 |
| pyrazin-2-yl]-phenol | | | | | |
| (5-Naphthalen-2-yl-pyrazin- | 326.4 | M + 1, | 327 | 368 | 653 |
| 2-yl)-(2-pyridin-3-yl-ethyl)- | | M + Acetonitrile + 1, | | | |
| amine | | 2M + 1 | | | |
| 4-Amino-N-{4-[5-(3-chloro- | 416.9 | M + 1 | 417 | | |
| pyridin-4-yl)-pyrazin-2- | | | | | |
| ylamino]-phenyl}-benzamide | | | | | |
| 4-[(5-Pyridin-4-yl-pyrazin-2- | 341.4 | M + 1 | 342 | | |
| ylamino)-methyl]- | | | | | |
| benzenesulfonamide | | | | | |
| [3,4′]Bipyridinyl-5-yl- | 311.4 | M + 1, | 312 | | 353 |
| naphthalen-2-ylmethyl-amine | | M + Acetonitrile + 1 | | | |
| 4-{5-[(Naphthalen-2- | 326.4 | M + 1, 2M + 1 | 327 | | 653 |
| ylmethyl)-amino]-pyridin-3- | | | | | |
| yl}-phenol | | | | | |
| 3-[5-(3,4-Dichloro- | 372.3 | M + 1, | 373 | 414 | 745 |
| benzylamino)-pyridin-3-yl]- | | M + Acetonitrile + 1, | | | |
| benzamide | | 2M + 1 | | | |
| [3,4′]Bipyridinyl-5-yl-(3,4- | 330.2 | M + 1 | 331 | | |
| dichloro-benzyl)-amine | | | | | |
| 4-[5-(3,4-Dichloro- | 345.2 | M + 1, | 346 | 387 | 691 |
| benzylamino)-pyridin-3-yl]- | | M + Acetonitrile + 1, | | | |
| phenol | | 2M + 1 | | | |
| 3-[5-(4-Chloro-benzylamino)- | 337.8 | M + 1, 2M + 1 | 338 | | 675 |
| pyridin-3-yl]-benzamide | | | | | |
| [3,4′]Bipyridinyl-5-yl-(4- | 295.8 | M + 1, | 296 | 337 | |
| chloro-benzyl)-amine | | M + Acetonitrile + 1 | | | |
| 4-[5-(4-Chloro-benzylamino)- | 310.8 | M + 1 | 311 | | |
| pyridin-3-yl]-phenol | | | | | |
| 3-[5-(3,4-Difluoro- | 339.3 | M + 1, | 340 | 381 | 679 |
| benzylamino)-pyridin-3-yl]- | | M + Acetonitrile + 1, | | | |
| benzamide | | 2M + 1 | | | |
| [3,4′]Bipyridinyl-5-yl-(3,4- | 297.3 | M + 1, | 298 | 339 | |
| difluoro-benzyl)-amine | | M + Acetonitrile + 1 | | | |
| 4-[5-(3,4-Dimethoxy- | 336.4 | M + 1, 2M + 1 | 337 | | 673 |
| benzylamino)-pyridin-3-yl]- | | | | | |
| phenol | | | | | |
| 3-{5-[(Benzo[1,3]dioxol-5- | 347.4 | M + 1, 2M + 1 | 348 | | 695 |
| ylmethyl)-amino]-pyridin-3- | | | | | |
| yl}-benzamide | | | | | |
| Benzo[1,3]dioxol-5-ylmethyl- | 305.3 | M + 1 | 306 | | |
| [3,4′]bipyridinyl-5-yl-amine | | | | | |
| 4-{5-[(Benzo[1,3]dioxol-5- | 320.4 | M + 1, 2M + 1 | 321 | | 641 |
| ylmethyl)-amino]-pyridin-3- | | | | | |
| yl}-phenol | | | | | |
| 4-[5-(3,4-Difluoro- | 312.3 | M + 1, | 313 | 354 | |
| benzylamino)-pyridin-3-yl]- | | M + Acetonitrile + 1 | | | |
| phenol | | | | | |
| 3-[5-(3-Hydroxy- | 363.4 | M + 1, 2M + 1 | 364 | | 727 |
| benzylamino)-pyridin-3-yl]- | | | | | |
| N-(2-hydroxy-ethyl)- | | | | | |
| benzamide | | | | | |
| 3-[5-(3-Hydroxy- | 319.4 | M + 1, 2M + 1 | 320 | | 639 |
| benzylamino)-pyridin-3-yl]- | | | | | |
| benzamide | | | | | |
| 3-[(5-Benzo[1,3]dioxol-5-yl- | 320.4 | M + 1, 2M + 1 | 321 | | 641 |
| pyridin-3-ylamino)-methyl]- | | | | | |
| phenol | | | | | |
| 3-([3,4′]Bipyridinyl-5- | 277.3 | M + 1 | 278 | | |
| ylaminomethyl)-phenol | | | | | |
| 3-{[5-(4-Hydroxymethyl- | 306.4 | M + 1, 2M + 1 | 307 | | 613 |
| phenyl)-pyridin-3-ylamino]- | | | | | |
| methyl}-phenol | | | | | |
| N-{3-[5-(3-Hydroxy- | 333.4 | M + 1, 2M + 1 | 334 | | 667 |
| benzylamino)-pyridin-3-yl]- | | | | | |
| phenyl}-acetamide | | | | | |
| (5-(4′-hydroxy-phenyl)- | 292.3 | M + 1, 2M + 1 | 293 | | 585 |
| pyridin-3-yl)-(3-hydroxy- | | | | | |
| benzyl)-amine | | | | | |
| 3-{[5-(3-Hydroxymethyl- | 306.4 | M + 1, 2M + 1 | 307 | | 613 |
| phenyl)-pyridin-3-ylamino]- | | | | | |
| methyl}-phenol | | | | | |
| (5-(3′-hydroxy-phenyl)- | 292.3 | M + 1 | 293 | | |
| pyridin-3-yl)-(3-hydroxy- | | | | | |
| benzyl)-amine | | | | | |
| 3-{5-[(Furan-3-ylmethyl)- | 293.3 | M + 1, | 294 | 335 | |
| amino]-pyridin-3-yl}- | | M + Acetonitrile + 1 | | | |
| benzamide | | | | | |
| [3,4′]Bipyridinyl-5-yl-furan-3- | 251.3 | M + 1 | 252 | | |
| ylmethyl-amine | | | | | |
| 4-{5-[(Furan-3-ylmethyl)- | 266.3 | M + 1 | 267 | | |
| amino]-pyridin-3-yl}-phenol | | | | | |
| 3-{5-[(Pyridin-3-ylmethyl)- | 304.4 | M + 1, 2M + 1 | 305 | | 609 |
| amino]-pyridin-3-yl}- | | | | | |
| benzamide | | | | | |
| [3,4′]Bipyridinyl-5-yl-(3- | 281.7 | M + 1, | 282 | 323 | |
| chloro-phenyl)-amine | | M + Acetonitrile + 1 | | | |
| 3-[5-(3-Bromo- | 368.2 | M + 1, | 369 | 410 | 737 |
| phenylamino)-pyridin- | | M + Acetonitrile + 1, | | | |
| 3-yl]-benzamide | | 2M + 1 | | | |
| [3,4′]Bipyridinyl-5-yl-(3- | 326.2 | M + 1 | 327 | | |
| bromo-phenyl)-amine | | | | | |
| [3,4′]Bipyridinyl-5-yl-(3- | 292.3 | M + 1, | 293 | 334 | |
| nitro-phenyl)-amine | | M + Acetonitrile + 1 | | | |

| Compound | mol weight | MS data | M + 1 | M + Cl | M+ [symbol] |
|---|---|---|---|---|---|
| 3-[(3′-Chloro-[3,4′]bipyridinyl-5- | 312 | M+ (35Cl), M+ (37Cl) | | 312 | |
| ylamino)-methyl]-phenol | | | | | |
| 3-[(5-Quinolin-5-yl-pyridin-3-ylamino)- | 327 | M + 1 | 328 | | |
| methyl]-phenol | | | | | |
| 3-{[5-(1H-Pyrazol-3-yl)-pyridin-3- | 266 | M + 1 | 267 | | |
| ylamino]-methyl}-phenol | | | | | |
| 3-[(5-Isoquinolin-4-yl-pyridin-3-ylamino)- | 327 | M + 1 | 328 | | |
| methyl]-phenol | | | | | |
| 4-[5-(3-Hydroxy-benzylamino)-pyridin-3- | 319 | M + 1 | 320 | | |
| yl]-benzamide | | | | | |
| 3-{[5-(4-Amino-phenyl)-pyridin-3- | 291 | M + 1 | 292 | | |
| ylamino]-methyl}-phenol | | | | | |
| 3-{[5-(1H-Indol-5-yl)-pyridin-3-ylamino]- | 315 | M + 1 | 316 | | |
| methyl}-phenol | | | | | |
| 3-[(5′-Methoxy-[3,3′]bipyridinyl-5- | 307 | M + 1 | 308 | | |
| ylamino)-methyl]-phenol | | | | | |
| 3-[(5-Phenyl-pyridin-3-ylamino)-methyl]- | 276 | M + 1 | 277 | | |
| phenol | | | | | |
| 3-{[5-(3-Amino-phenyl)-pyridin-3- | 291 | M + 1 | 292 | | |
| ylamino]-methyl}-phenol | | | | | |
| 5-{[5-(4-Hydroxy-phenyl)-pyridin-3- | 308 | M + 1 | 309 | | |
| ylamino]-methyl}-benzene-1,3-diol | | | | | |
| 3-[5-(3,5-Dihydroxy-benzylamino)- | 335 | M + 1 | 336 | | |
| pyridin-3-yl]-benzamide | | | | | |
| [3,4′]Bipyridinyl-5-yl-(3-chloro-benzyl)- | 296 | M+ (35Cl), M+ (37Cl) | | 296 | |
| amine | | | | | |
| 3-[5-(3-Chloro-benzylamino)-pyridin-3- | 338 | M+ (35Cl), M+ (37Cl) | | 338 | |
| yl]-benzamide | | | | | |
| [3,4′]Bipyridinyl-5-yl-(4-methoxy- | 291 | M + 1 | 292 | | |
| benzyl)-amine | | | | | |
| 4-[5-(4-Methoxy-benzylamino)-pyridin-3- | 306 | M + 1 | 307 | | |
| yl]-phenol | | | | | |
| 5-{[5-(4-Hydroxy-phenyl)-pyridin-3- | 322 | M + 1 | 323 | | |
| ylamino]-methyl}-2-methoxy-phenol | | | | | |
| 3-[5-(3-Hydroxy-4-methoxy- | 349 | M + 1 | 350 | | |
| benzylamino)-pyridin-3-yl]-benzamide | | | | | |
| 3-([3,4′]Bipyridinyl-5-ylamino)-phenol | 263 | M + 1 | 264 | | |
| 4-[5-(3-Hydroxy-phenylamino)-pyridin-3- | 278 | M + 1 | 279 | | |
| yl]-phenol | | | | | |
| 3-[5-(3-Hydroxy-phenylamino)-pyridin-3- | 305 | M + 1 | 306 | | |
| yl]-benzamide | | | | | |
| [3,4′]Bipyridinyl-5-yl-cyclohexylmethyl- | 267 | M + 1 | 268 | | |
| amine | | | | | |
| 4-[5-(Cyclohexylmethyl-amino)-pyridin- | 282 | M + 1 | 283 | | |
| 3-yl]-phenol | | | | | |
| 3-[5-(Cyclohexylmethyl-amino)-pyridin- | 309 | M + 1 | 310 | | |
| 3-yl]-benzamide | | | | | |
| [3,4′]Bipyridinyl-5-yl-(4-chloro-3-fluoro- | 314 | M+ (35Cl), M+ (37Cl) | | 314 | |
| benzyl)-amine | | | | | |
| 3-[5-(4-Chloro-3-fluoro-benzylamino)- | 356 | M+ (35Cl), M+ (37Cl) | | 356 | |
| pyridin-3-yl]-benzamide | | | | | |
| [3,4′]Bipyridinyl-5-yl-(3- | 345 | M + 1 | 346 | | |
| trifluoromethoxy-benzyl)-amine | | | | | |
| 4-[5-(3-Trifluoromethoxy-benzylamino)- | 360 | M + 1 | 361 | | |
| pyridin-3-yl]-phenol | | | | | |
| 3-[5-(3-Trifluoromethoxy-benzylamino)- | 387 | M + 1 | 388 | | |
| pyridin-3-yl]-benzamide | | | | | |
| 3-[5-(4-Hydroxy-benzylamino)-pyridin-3- | 319 | M + 1 | 320 | | |
| yl]-benzamide | | | | | |
| 3-{5-[(Pyrrolidin-2-ylmethyl)-amino]- | 296 | M + 1 | 297 | | |
| pyridin-3-yl}-benzamide | | | | | |
| 4-{5-[(Pyrrolidin-2-ylmethyl)-amino]- | 269 | M + 1 | 270 | | |
| pyridin-3-yl}-phenol | | | | | |
| [3,4′]Bipyridinyl-5-yl-pyrrolidin-2- | 254 | M + 1 | 255 | | |
| ylmethyl-amino | | | | | |
| 4-[5-(3-Chloro-benzylamino)-pyridin-3- | 311 | M+ (35Cl), M+ (37Cl) | | 311 | |
| yl]-phenol | | | | | |
| N-{4-[5-(3-Hydroxy-benzylamino)- | 333 | M + 1 | 334 | | |
| pyridin-3-yl]-phenyl}-acetamide | | | | | |
| 4-{5-[(3-hydroxy-benzyl)-methyl-amino]- | 306 | M + 1 | 307 | | |
| pyridin-3-yl}-phenol | | | | | |
| 3-[5-(3-Hydroxy-benzylamino)-pyridin-3- | 363 | M + 1 | 364 | | |
| yl]-N-(2-hdroxy-ethyl)-benzamide | | | | | |
| 3-[5-(4-Methoxy-benzylamino)-pyridin-3- | 333 | M + 1 | 334 | | |
| yl]-benzamide | | | | | |
| N-[3,4′]Bipyridinyl-5-yl-2-(4-chloro- | 324 | M+(35Cl), M+ (37Cl) | | 324 | |
| phenyl)-acetamide | | | | | |
| [3,4′]Bipyridiny-5-yl-(4-bromo-benzyl)- | 340 | M+ (79Br), M+ (81Br) | | | 34 [symbol] |
| amine | | | | | |

[symbol]

indicates data missing or illegible when filed

It will be appreciated by those skilled in the art that the foregoing description is exemplary and explanatory in nature, and is intended to illustrate the invention and its preferred embodiments. Through routine experimentation, an artisan will recognise apparent modifications and variations that may be made without departing from the spirit of the invention. Thus, the invention is intended to be defined not by the above description, but by the following claims and their equivalents.

1. A compound selected from the group of compounds consisting of compounds of formula (I) and compounds of formula (II): 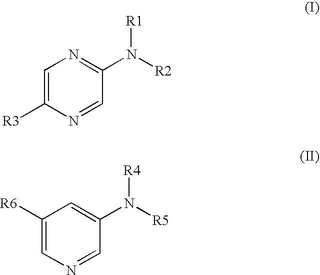
wherein:
R1 and R2 are joined to form a ring system; or
R1 is H; and
R2 is 2-pyridin-4-yl-ethyl; 3-chloro-benzyl; benzo[1,3]dioxol-4-ylmethyl; 4-sulfonamide-benzyl; benzyl; thiophen-2-ylmethyl; 1-phenyl-ethyl; 4-(4-amino-benzoylamino)-phenyl; 4-methoxy-benzyl; 1-hydroxymethyl-2-methyl-propyl; 2-Pyridin-3-yl-ethyl; 4-phenoxy-phenyl; 4-fluoro-phenyl; 4-[ethyl-(2-hydroxy-ethyl)-amino]-phenyl; C1-C6 optionally substituted alkyl, C3-C6 optionally substituted cycloalkyl; 5 to 7 membered optionally substituted containing 1 to 3 heteroatoms selected from nitrogen and oxygen, R2 is optionally linked to the scaffold by a linker which includes 1 to 3 carbon atoms;
R3 is benzofuran-2-yl; naphthalen-2-yl; 3-4-methoxy-phenyl; 4-thiomethyl-phenyl; benzothiophen-2-yl; 4-pyridyl; 4-methoxy-phenyl; quinolin-3-yl; benzo[1,3]dioxol-5-yl; 4-hydroxy-phenyl; 4-trifluoromethoxy-phenyl; 3-chloro-4-pyridyl; 3-4-5-methoxy-phenyl; 5-acetyl-thiophen-2-yl; 3-trifluoromethoxy-phenyl; 4-hydroxymethyl-phenyl; N-(4-Methoxy-phenyl)-benzamide-4-yl; 3-fluoro-4-chloro-phenyl; N-(2-Hydroxy-ethyl)-4-benzamide-4-yl; 3-hydroxy-phenyl; 3-acetylamino-phenyl; quinolin-7-yl; 2-methoxy-5-isopropyl-phenyl; 3-hydroxymethyl-phenyl; 3-pyridyl; hex-1-enyl; 4-cyano-phenyl; thiophen-3-yl; 3-nitro-phenyl; 3-chloro-phenyl; 2-methoxy-phenyl; 4-isopropyl-phenyl;
R4 and R5 are joined to form a ring system; or
R4 is H or methyl; and
R5 is 3-hydroxy-phenyl; 3-hydroxybenzoyl; 4-bromo-benzyl; 4-methoxybenzyl; 2,5-hydroxybenzyl; 3-hydroxy-4-methoxy-benzyl; 3-chloro-benzyl; 3-fluoro-4-chloro-benzyl; 3-amino-benzyl; 3-trifluoromethoxy-benzyl; 4-hydroxy-benzyl; 4-amino-benzyl; 1H-Indol-6-yl; 3-hydroxy-benzyl; naphthalen-2-yl-methyl; benzo[1,3]dioxol-4-ylmethyl; 3,4-fluoro-benzyl; 3,4-chloro-benzyl; furan-3-yl-methyl; 4-methoxy-phenyl; 4-chloro-benzyl; 3-nitro-phenyl; 3,4-methoxy-phenyl; 3-bromo-phenyl; 4-chloro-phenyl; phenyl; 3-chloro-phenyl; 2-naphtyl; pyridin-3-yl-methyl; pyridin-4-yl-methyl; quinolin-3-yl-methyl; 4-isopropyl-phenyl; 4-chloro-benzyl; 3,4-methoxy-benzyl; 3-fluoro-4-chloro-phenyl; 4-trifluoromethoxy-phenyl; 4-cyano-phenyl; 4-metoxy benzyl, 4-methoxy-3-hydroxy benzyl; pyridin-4-yl-ethyl; piperidine-1-carboxylic acid benzyl ester 3-yl-methyl ; cyclohexane-methyl; 4-chlorobenzoyl; pyrrolidine-2-yl-methyl; C1-C6 optionally substituted alkyl, C5-C7 optionally substituted cycloalkyl; 5 to 7 membered optionally substituted containing 1 to 3 heteroatoms selected from nitrogen and oxygen, and R5 is optionally linked to the scaffold by a linker which includes 1 to 3 carbon atoms; and
R6 is 3-carbamoyl-phenyl; 4-hydroxy-phenyl; 4-amino-phenyl; 3-amino-phenyl; phenyl; 1H-Indol-5-yl; 4-pyridyl; 3-hydoxy-phenyl; Benzo[1,3]dioxol-5-yl; 3-(2-Hydroxy-ethylcarbamoyl)-phenyl; 3-hydroxymethyl-phenyl; 3-acetylamino-phenyl; 4-hydroxymethyl-phenyl; 3-(2-dimethylamino-ethylcarbamoyl)-phenyl; thiophene-3-yl; 3-pyridyl; 3,4-methoxy-phenyl; 6-Bromo-1-carboxylic acid tert-butyl ester-indol-2-yl; 3-(2-hydroxy-ethylcarbamoyl)-phenyl; 3-Methanesulfonylamino-phenyl; 3-trifluoromethoxy-phenyl; 4-hydroxymethyl-phenyl; 4-methanesulfonyl-phenyl; quinolin-3-yl; 5-methoxy-pyridin-3-yl;4-carbamoyl-phenyl; 4-acetylamino-phenyl; 4-Methylcarbamoyl-phenyl; 4-(2-Hydroxy-ethylcarbamoyl)-phenyl; quinolin-4-yl; quinolin-5-yl; isoquinolin-4-yl; 1H-pyrazol-4-yl; 3-chloro-pyridin-4-yl; 3-methoxy-pyridin-5-yl; 4-methoxy-pyridin-5-yl; or 2-methyl-pyridin-4-yl; benzothiophene-2-yl; 3-chloro-pyridine-4-yl; 1H-pyrazol-3-yl; isoquinolin-3-yl; 4-carbamoyl-phenyl; 4-carbamoyl-phenyl; 3-(2-Hydroxy-ethylcarbamoyl)-phenyl.

2. The compound according to claim 1, wherein R1 and R2 form a 5 to 7 membered ring optionally substituted containing 1 to 3 heteroatoms selected from nitrogen and oxygen.

3. The compound according to claim 2 wherein the ring is selected from the group consisting of 2-(2-hydroxy-ethyl)-piperidin-1-yl or 4-(2-hydroxy-ethyl)-piperazin-1-yl; 4-methyl-piperazin-1-yl; 4-pyridin-4-yl-piperazin-1-yl; 4-(2-dimethylamino-ethyl)-piperazin-1-yl; 4-(2-diethylamino-ethyl)-piperazin-1-yl; morpholin-4-yl; 4-(2-cyano-phenyl)-piperazin-1-yl; 4-methyl-[1,4]diazepan-1-yl; N-(2-dimethylamino-ethyl)-N-methyl-; 4-(3,4-dimethoxy-phenyl)-piperazin-1-yl; 4-pyridin-2-yl-piperazin-1-yl; 4-(2-hydroxy-ethyl)-piperazin-1-yl; 4-(furan-3-carbonyl)-piperazin-1-yl; 4-(2-pyrrolidin-1-yl-ethyl)-piperazin-1-yl and 2-(2-hydroxy-ethyl)-pyrrolidin-1-yl.

4. The compound according to claim 1 wherein R4 and R5 form a 5 to 7 membered ring optionally substituted containing 1 to 3 heteroatoms selected from nitrogen and oxygen.

5. The compound according to claim 4 wherein the ring is selected from the group consisting of 2-(2-hydroxy-ethyl)-piperidin-1-yl or 4-(2-hydroxy-ethyl)-piperazin-1-yl; 4-methyl-piperazin-1-yl; 4-pyridin-4-yl-piperazin-1-yl; 4-(2-dimethylamino-ethyl)-piperazin-1-yl; 4-(2-diethylamino-ethyl)-piperazin-1-yl; morpholin-4-yl; 4-(2-cyano-phenyl)-piperazin-1-yl; 4-methyl-[1,4]diazepan-1-yl; N-(2-dimethylamino-ethyl)-N-methyl-; 4-(3,4-dimethoxy-phenyl)-piperazin-1-yl; 4-pyridin-2-yl-piperazin-1-yl; 4-(2-hydroxy-ethyl)-piperazin-1-yl; 4-(furan-3-carbonyl)-piperazin-1-yl; 4-(2-pyrrolidin-1-yl-ethyl)-piperazin-1yl and 2-(2-hydroxy-ethyl)-pyrrolidin-1-yl.

6. The compound according to claim 1, wherein:
R1 is hydrogen;
R2 is 2-pyridin-4-yl-ethyl; thiophen-2-ylmethyl; 4-sulfonamide-benzyl; or 3-chloro-benzyl;
R3 is benzothiophen-2-yl; naphthalen-2-yl; 3-4-methoxy-phenyl; or 4-pyridyl;
R4 is hydrogen;
R5 is 3-hydroxy-benzyl; 4-chloro-benzyl; naphthalen-2-yl-methyl;
benzo[1,3]dioxol-4-ylmethyl; 3,4-fluoro-benzyl; 3,4-chloro-benzyl; or furan-3-yl-methyl; and
R6 is 3-carbamoyl-phenyl; 4-hydroxy-phenyl; or 4-pyridyl.

7. The compound according to claim 1 that is selected from the group of compounds named in Table A or Table B.

8. A method for making a compound according to claim 1, which method comprises at least one step or a series of consecutive steps from the scheme defined herein.

9. A group of two or more compounds comprising two or more compounds of claim 1.

10. The group of two or more compounds according to claim 9 wherein all or substantially all of the permitted substitutions of formula (I) and formula (II) are represented by compounds in said group.

11. A method for making a group of compounds according to claim 10, which method comprises at least one step or a series of consecutive steps from the scheme defined herein.

12. An assay comprising a compound according to claim 1.

13. (canceled)

14. A pharmaceutical composition that comprises a compound according to claim 1 and a pharmaceutically acceptable carrier.

15-17. (canceled)

18. A method of treatment of a condition characterised by abnormal kinase activity that comprises administering a pharmaceutically effective amount of a compound according to claim 1.

19. The method of treatment according to claim 18 wherein the condition is selected from cardiovascular disease stroke, cancer, erectile dysfunction, asthma, osteoporosis, glaucoma and AIDS.

20. (canceled)

21. The compound according to claim 1, wherein R2 is a C1-C6 optionally substituted alkyl, C3-C6 optionally substituted cycloalky, or 5 to 7 membered optionally substituted ring containing 1 to 3 heteroatoms selected from nitrogen and oxygen, wherein
said C1-C6 optionally substituted alkyl is selected from the group consisting of ethyl, propyl, 3-hydroxy-2,2-dimethyl-propyl, 3-hydroxy-propyl, 2-methoxy-ethyl, 2-hydroxy-ethyl, 2-hydroxymethyl-3-methyl-butyl, 1-hydroxymethyl-propyl, 2-morpholin-4-yl-ethyl, and furan-2-yl-methyl;
said C3-C6 optionally substituted cycloalky is optionally substituted cyclohexyl; and
said 5 to 7 membered optionally substituted ring containing 1 to 3 heteroatoms selected from nitrogen and oxygen is optionally substituted piperazinyl, optionally substituted [1,4]diazepanyl or optionally substituted pyrrolidinyl.

22. The compound according to claim 1, wherein R2 is a C1-C6 optionally substituted alkyl, C5-C7 optionally substituted cycloalky, or 5 to 7 membered optionally substituted ring containing 1 to 3 heteroatoms selected from nitrogen and oxygen, wherein
said C1-C6 optionally substituted alkyl is selected from the group consisting of ethyl, propyl, 3-hydroxy-2,2-dimethyl-propyl, 3-hydroxy-propyl, 2-methoxy-ethyl, 2-hydroxy-ethyl, 2-hydroxymethyl-3-methyl-butyl, 1-hydroxymethyl-propyl, 2-morpholin-4-yl-ethyl, and furan-2-yl-methyl;
said C5-C7 optionally substituted cycloalky is optionally substituted cyclohexyl; and
said 5 to 7 membered optionally substituted ring containing 1 to 3 heteroatoms selected from nitrogen and oxygen is optionally substituted piperazinyl, optionally substituted [1,4]diazepanyl or optionally substituted pyrrolidinyl.

23. The method of claim 19 wherein the condition is cardiovascular disease and said cardiovascular disease is selected from the group consisting of coronary vasospasm, hypertensive disease and arteriosclerosis.

24. A pharmaceutical composition that comprises a compound according to claim 6 and a pharmaceutically acceptable carrier.

25. A pharmaceutical composition that comprises a compound according to claim 7 and a pharmaceutically acceptable carrier.
